(19) United States
(12) Patent Application Publication
(10) Pub. No.: US 2020/0291470 A1
(43) Pub. Date: Sep. 17, 2020

(54) SEQUENCING NUCLEIC ACID SEQUENCES

(71) Applicant: HEWLETT-PACKARD DEVELOPMENT COMPANY, L.P., Spring, TX (US)

(72) Inventors: Adam Higgins, Corvallis, OR (US); Viktor Shkolnikov, Palo Alto, CA (US); Jeffrey A. Nielsen, Corvallis, OR (US)

(73) Assignee: HEWLETT-PACKARD DEVELOPMENT COMPANY, L.P., Spring, TX (US)

(21) Appl. No.: 16/645,223

(22) PCT Filed: Oct. 13, 2017
(86) PCT No.: PCT/US2017/056579

§ 371 Date: Mar. 6, 2020

(51) Int. Cl. C12Q 1/6874; B01L 3/02; C12Q 1/6806

(52) U.S. Cl. CPC C12Q 1/6874; B01L 3/0268; C12Q 1/6806; B01L 2200/0647; C12Q 2527/146; B01L 2300/0645; B01L 2400/0439; B01L 2400/0442; B01L 2200/143

(57) ABSTRACT

A method of sequencing multiple nucleic acid sequences, including: estimating a concentration of cells in a solution; and depositing a volume of the solution into a well on a well plate using an ejection device, the amount of solution selected to contain no more than a single cell.

### BACKGROUND

Sequencing genomic material may be performed using shotgun sequencing. This involves by fragmenting the sequence, sequencing the fragments, for example with chain termination sequencing or next generation sequencing, and then reconstructing the whole sequence from the overlaps between fragments. This limits the number of the cycles performed on a given fragment, which reduces the overall time to process a lengthy sequence. This approach also facilitates parallel processing and measurement. This approach may results in multiple measurements, e.g., five to twenty, for each base-pair in the sequence.

### BRIEF DESCRIPTION OF THE DRAWINGS

The accompanying drawings illustrate various examples of the principles described herein and are a part of the specification. The illustrated examples do not limit the scope of the claims.

FIG. 1 shows a flowchart for a method of sequencing multiple nucleic acid sequences consistent with the present specification.

FIG. 2 shows a system for sequencing a plurality of nucleic acid sequences consistent with the present specification

FIG. 3 shows a system for sequencing multiple nucleic acid sources consistent with the present specification.

FIG. 4 shows a cross-sectional view through a pair of nozzles of two different firing chambers in an example of an ejection device consistent with the present specification.

Throughout the drawings, identical reference numbers designate similar, but not necessarily identical, elements. The figures are not necessarily to scale, and the size of some parts may be exaggerated to more clearly illustrate the example shown. Moreover the drawings provide examples and/or implementations consistent with the description; however, the description is not limited to the examples and/or implementations provided in the drawings.

### DETAILED DESCRIPTION

Shotgun sequencing was a useful advance to sequencing of nucleic acid sequences, for example, DNA and/or RNA sequences. The ability to run sequencing operations in parallel rather than series drastically reduces the time to process longer sequences.

Some populations to be sequenced include more than one nucleic acid sequence. For example, it may be desired to sequence a population of microorganisms. One could process the entire population using shotgun sequencing. However, the difficulty of reconstructing the fragments into unique sequences becomes increasingly challenging as the number of organisms increases.

As the size of fragments being sequenced decreases, the time to sequence a given fragment also decreases. However, shorter fragments increase the number of matches to assemble the entire sequence. Shorter fragments also contain less information in the overlap, increasing the probability that some overlaps will be the same, resulting in ambiguity. Nevertheless increasing computing power to reconstruct the sequence allows use of smaller fragments with shorter processing times and higher throughput.

Trying to process multiple sequences simultaneously produces additional difficulties. The multiple sequences may have commonalities in part of the sequence. For example, when trying to sequence a microbiome, some microbes may have identical and/or similar runs. This can make reconstruction of such a complex set of fragments challenging. As an analogy, if putting together a single sequence is similar to assembling a jigsaw puzzle, then putting together a set of sequences is assembling a set of jigsaw puzzles that have been mixed together. Heterogeneity in the biome will make this operation easier, just like different pictures on the puzzles may make solving a set of puzzles easier, However, similar sequences in a biome make reconstruction harder, just like two puzzles of similar subject matter make solving the mixed jigsaw puzzles more challenging.

One solution used is to label the various sequences, for example with radio nuclei, fluorescent, and/or chemical tags. However, this is labor intensive and increases the cost of such technologies. There are also a finite number of discrete tags that can be used. Further, some labeling approaches may limit and/or impede sequencing the fragments.

One challenge with dealing with solutions containing multiple sequences is separating sequences from each other to prevent confounding, An estimate of the concentration of nucleic acid sequences and/or the parent cells in a solution may be made. This may be performed using absorbance for instance, This may be a chemical measurement. This concentration is used to determine a volume per sequence, which is to say, an inverse of concentration. Next, a fraction of this volume is determined to minimize the capture of two sequences in a sample, For example, if the volume per cell was 50 picoliters, a fraction of 10% may be used for a target sample size of 5 picoliters. In a similar approach, the sample may be diluted or concentrated to obtain a desired sequence probability in a predetermined droplet size provided by an ejector. Dilution may be performed with a saline solution. Dilution may be performed with a solution which contains species for a process to be performed on the droplet.

The solution is provided to an ejection device which includes an array of ejectors. The ejectors deposit droplets of the solution into wells on a plate. The wells may be assessed to determine the presence or absence of a target, such as a cell or a nucleic acid sequence, in a given well. If a well lacks a target, an additional droplet can be deposited using the ejection device. This can be repeated until a maximum number of droplets are reached, a time limit is reached, and/or another limit condition.

In an example, the wells include a transparent bottom which allows light based detection of a nucleic acid sequence and/or a cell. The location of the detection device underneath the wells keeps the detection device clear of the deposition and fluid handling performed through the top of the well. In an example, the system includes a camera to detect the presence or absence of a cell in a well. However, UV-VIS spectroscopy may be used due to the strong absorbance of nucleic acids in the 260 nm range.

The ability to verify the presence of a cell and/or nucleic acid sequence for evaluation also allows the application of multiple droplets without the risk of having multiple cells by using droplets sized to be unable to contain two or more cells. For example, if the volume is 10% of the volume per cell. Then, statistically, the first pass will put cells on 10% of the wells. The next pass will put cells on 10% of the remaining 90% of the wells for an additional 9% of intake ports being loaded. Each pass has incrementally fewer wells loaded as already loaded wells are excluded, resulting in diminishing returns. However, the ability to rapidly apply a droplet to selected wells, based on feedback, allows use of more wells on the device without cross talk from multiple sequences in a well. This higher usage, in turn, reduces the per sample cost. For example, five passes with a 10% well loading rate produces 41% loading. This is four times the usage of a single pass device. And this efficiency is achieved due to the ability to rapidly deposit droplets and the ability to detect loaded wells using some technique. Optical techniques provide high speed and parallel evaluation of a large number of wells. The processing is also amenable to parallel processing where small portions of the optical image are distributed and processed separately, These features enable rapid loading of a well plate.

Among other examples, this specification describes a method of sequencing multiple nucleic acid sequences, including: estimating a concentration of cells in a solution; and depositing a volume of the solution into a well on a well plate using an ejection device, the amount of solution selected to contain no more than a single cell.

This specification also describes a system for sequencing a plurality of nucleic acid sequences, the system including: an ejection device comprising a reservoir, the reservoir to hold a solution containing a plurality of cells, the plurality of cells containing different respective nucleic acid sequences to be sequenced; and a controller to receive a concentration of cells in the solution and to modify the deposited volume of the solution by the ejection device based on the concentration of cells in the solution.

This specification also describes a system for sequencing multiple nucleic acid sources, the system including: an ejection device with a reservoir; a sensor to detect absorption of light passing through the reservoir; and a controller to calculate a cell density in a solution in the reservoir from an output of the sensor, wherein the ejector device receives solution from the reservoir and ejects solution as droplets, each droplet sized to contain at most a single cell, the droplet size being determined by the controller.

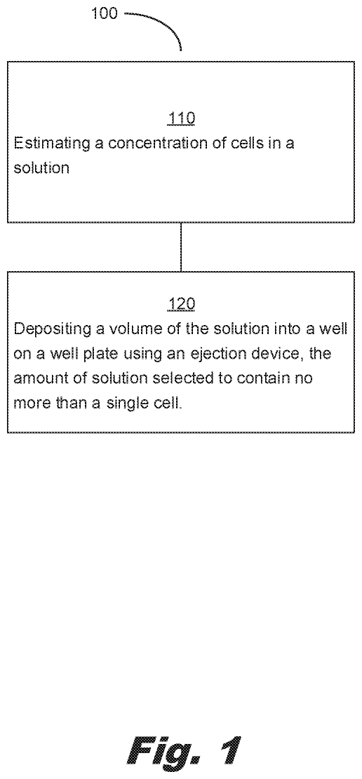

Turning now to the drawings, FIG. 1 shows a flowchart for a method (100) of sequencing multiple nucleic acid sequences. The method (100) includes (110) estimating a concentration of cells in a solution; and (120) depositing a volume of the solution into a well on a well plate using an ejection device, the amount of solution selected to contain no more than a single cell.

The method (100) includes estimating a concentration of cells in a solution (110). The concentration of cells in the solution may be estimated using a variety of approaches. For example, the Beer-Lambert law can be used to estimate the concentration of cells using transmission of light through the solution. Optical density measurements provide another method. An indicator, marker, and/or tag may be added to the solution and a property associated with the indicator, marker, and/or tag measured, e.g., fluorescence.

The method (100) includes depositing a volume of the solution into a well on a well plate using an ejection device, the amount of solution selected to contain no more than a single cell (120). This may include determining an average volume per cell in the solution, which is the inverse of concentration, A droplet volume can then be selected based on this volume per cell which minimizes the chance of multiple cells in a droplet. The droplet volume may be from 1% to 20% of the volume per cell. In some examples the droplet volume is from 8% to 12% of the volume per cell. The droplet volume may be between 5 and 100 picoliters. In some examples the droplet volume is between 20 and 60 picoliters.

The method may also include: depositing lysing reagent into the well using the ejection device. Again the rapid deposition by the ejection device enables larger numbers of wells to be processed simultaneously. The method (100) may include heating the well.

The method (100) may include removing reacted reagent from the well, This may be performed with a robotic pipette or multipipette. Removal of the used materials such as reacted reagents may be performed in multiple wells simultaneously. This may decrease the total process time. Reacted materials may also be removed by vacuum. In an example, a vacuum removes reacted materials from multiple wells simultaneously, The vacuum may traverse from one side of the well plate to the other. The vacuum may come down over the entire well plate simultaneously. The vacuum may be used in combination with a rinse and/or fluid feed (e.g., an air blower) to produce the desired extraction from the wells.

The method (100) may include diluting the solution to obtain a desired deposition volume prior to depositing the volume of the solution using the ejection device. The method (100) may include concentrating the solution to obtain a desired deposition volume prior to depositing the volume of the solution using the ejection device. This approach has the advantage of decreasing the hardware variation to support the desired droplet concentration of cells. Instead, the solution concentration is modified to produce the desired average solution volume per cell (inverse concentration). In an example, the concentration of the solution is monitored as the concentration is adjusted until the desired concentration is reached. Clearly, assuring good mixing of the dilutant to avoid local concentrations is helpful when adjusting the concentration. Avoiding gas bubbles and similar features when can cause measurement errors in absorbance/transmission measurements is also desirable.

The method (100) may also include detecting the presence or absence of a cell in the deposited droplet. The method (100) may further include applying an additional droplet to wells that do not have a cell in the droplet in the well. These operations may be repeated until an end condition is reached. Some example end conditions include: a maximum number of droplets, a maximum time since the first droplet was ejected, and/or a minimum percentage of wells with a cell.

The cell may be detected optically, spectrographically, electrically, and/or using other methodologies. Optical methods are advantageous due to the ability to use a common sensor for cell detection and reading tags from sequencing. However, using multiple sensors and/or different types of sensors may provide greater selectivity. Accordingly, depending on the design specification for the system, different optimums may exist.

The following workflow shows a number of different processes which may be included in a variety of combinations:
1. Using benchtop spectrophotometer, estimate the concentration of cells of interest (e.g., bacteria) in the sample, based on an absorption (turbidity) measurement. Let the concentration of cells be N.
2. Use ejection device to portion the sample in at least N volumes (preferentially 10*N) volumes, guaranteeing that each volume contains no more than a single cell of interest, and dispense these into individual wells. The well plate may be a standard multiwall plate (e.g. a 1536 well plate) with primers attached to the bottom surfaces. The resulting volumes may be fairly small (e.g., 10 pL). Such small volumes are difficult to obtain without an ejectors system. Manual and robotic processes are time consuming and have difficulty applying picoliter scale droplets. Hence the ejection device is central to making this process function.
3. Provide lysing reagent into all wells.
4. Heat the well plate to induce lysing in all wells. Lysing reagents fragment the DNA.
5. Provide ligation reagents including ligation adapters into all wells. The amount of ligation adapters should be less than the amount of primers attached to the bottom of the well.
6. Heat the well plate to appropriate temperature for the ligation reaction to occur.
7. Lower temperature to allow ligated DNA of interest to bind to the primers attached to the bottom of the well. Allow the ligated DNA to undergo bridge amplification.
8. Withdraw all solution from the wells.
9. Add wash solution to the wells.
10. Withdraw all solution from the well.
11. Provide fluorescent labelled dNTPs to the wells.
12. Raise temperature to allow the polymerization reaction (DNA extension) to occur.
12. Withdraw all solution from the well, washing the well.
13. Use fluorescence reader (e.g., a camera) to obtain fluorescence in the well. The fluorescence reader may be located under the well plate. The fluorescence reader may be located above the well plate. Record the sequence element based on the fluorescence signal.
14. Repeat to obtain DNA sequences in the well plate.

Providing the various solutions may be performed using the ejection device, a robotic system, manually, and/or some combination. The ejection system provides speed advantages. Similarly, withdrawing the used solutions from the wells may be performed using a variety of different devices including: a vacuum system, a pipetting system, robotic systems, manual systems, etc. Different system choices have tradeoffs including cost, throughput, and versatility, which is to say ability to conduct other tests using the same equipment.

Each well is limited to a single sequence; however, multiple fragments of that sequence are sequenced simultaneously at different attachment points on the bottom of a common well. Since all the fragments are from the same sequence in a well, the fragments from a given well can be combined with each other without having a risk of matching fragments from another sequence in another well. Further, when two fragments in different wells are matched (by overlap), that allows extrapolation to the other fragments in both of the wells. This knowledge that all the fragments in a given well sharing a common source sequence simplifies the process of sequencing a large number of different nucleic acid sequences simultaneously.

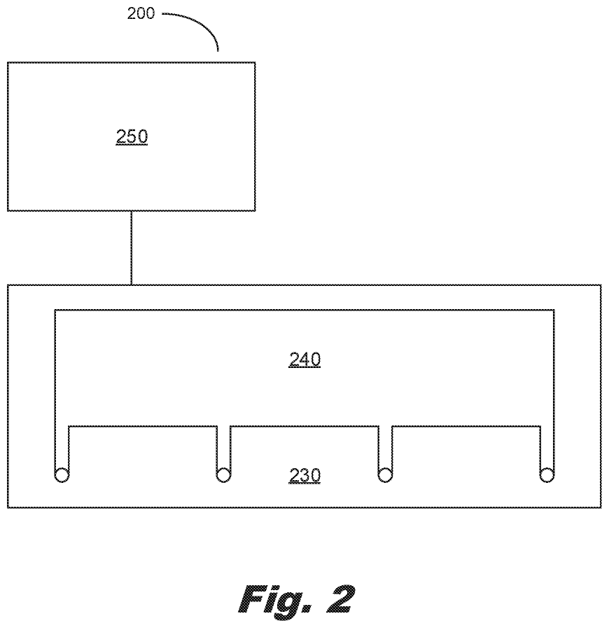

FIG. 2 shows a system (200) for sequencing a plurality of nucleic acid sequences, the system (200) including: an ejection device (230) including a reservoir (240), the reservoir (240) to hold a solution containing a plurality of cells, the plurality of cells containing different respective nucleic acid sequences to be sequenced; and a controller (250) to receive a concentration of cells in the solution and to modify the deposited volume of the solution by the ejection device (240) based on the concentration of cells in the solution.

The system (200) allows rapid loading of a well plate with ejected droplets, where each droplet is sized to limit its potential to contain more than one sequence to be sequenced. This may include limiting each droplet to a single cell. This may include droplets that contain DNA sequences. This may include droplets that contain RNA sequences.

The ejection device (230) uses an ejector to eject a droplet onto a well plate. The ejection device (230) may use a piezoelectric actuator. For example, the ejection device (230) may be a piezoelectric inkjet (PIJ). The ejection device (230) may use a heated gas bubble. For example, the ejection device (230) may be a thermal inkjet (TIJ).

The ejection device (230) may include a plurality of sizes of firing chambers and/or nozzles. In an example, the ejection device includes multiple banks of ejectors where each bank produces a different size droplet, Based on signals from the controller (250), the ejection device fires the bank of ejectors sized to provide a droplet which is unlikely to contain multiple nucleic acid sequences to be sequenced.

A firing chamber in the ejection device (230) may include more than one heater and/or actuator. For example, a firing chamber may include two heaters located such that activating the first heater ejects a droplet of a first volume and activating the second heater ejects a droplet of a second, different volume. Firing both the first and the second heaters may produce a droplet of a third volume. A firing chamber may include a piezoelectric actuator which ejects a droplet with a first volume and a heater which ejects a droplet of a second volume. This principle can be expanded to three, four, or more heaters and/or piezoelectric actuators allowing control over the ejected droplet size.

A firing chamber may include a sensor to detect the presence or absence of a cell in the volume of fluid to be ejected. In an example, the sensor is a light based sensor. The light may be provided locally to the firing chamber. The light may be provided generally to a bank of firing chambers, for example, by transmission through a wall of the firing chamber. The sensor may be a photoelectric sensor. The sensor may be wavelength specific or may detect a range of light.

The firing chamber may include an impedance sensor. The impedance sensor may operate between an electrode and a heater. The impedance sensor may operate between two electrodes. The impedance sensor may be located on a feed to the firing chamber. The impendence sensor may detect the passage of a cell based on the non-conductivity of the cell compared with the fluid surrounding the cell. The impedance sensor may support other firing chamber and/or nozzle diagnostics, for example, detection of a plugged nozzle.

The reservoir (240) contains a solution with nucleic acid sequences to be targeted. In an example, the nucleic acid sequences are the DNA sequences of individual cells. The solution may include cells. The solution may include free nucleic acid sequences. In an example, the solution includes a nucleic acid sequence that has been amplified and/or fragmented to produce a large number of clonal fragments from a single sequence, The fragments are then used for shotgun sequencing. Here the concentration of nucleic acid fragments and/or the droplet volume is controlled to limit each droplet to no more than a single fragment to be sequenced.

The controller (250) provide signals to the ejector device (230) to cause the ejector device (230) to eject a droplet of a desired size when the ejector device (230) is located such that the droplet will be deposited in a target well on the well plate. The combination of the controller (250) and ejector device (230) may have the ability to generate a variety of different sized droplets dynamically. The ejection device (230) may be selected based on concentration information provided to the controller (250). The concentration of the solution provided to the reservoir (240) of the ejector device (230) may be adjusted to a target range based on range of droplet sizes which can be produced by a given ejection device (230).

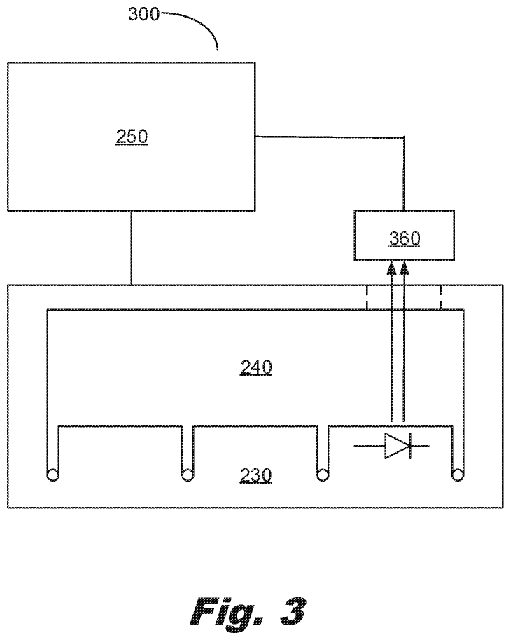

FIG. 3 shows a system (300) for sequencing multiple nucleic acid sources. The system (300) includes: an ejection device (230) with a reservoir (240); a sensor (360) to detect absorption of light passing through the reservoir (240); a controller (250) to calculate a cell density in a solution in the reservoir (240) from an output of the sensor (360); wherein the ejector device (230) receives solution from the reservoir (240) and ejects solution as droplets, each droplet sized to contain at most a single cell, the droplet size being controlled by the controller (250).

The sensor (360) detects absorption of light passing through the reservoir (240). In an example, the sensor includes a light source. A portion of the reservoir (240) wall may be reflective such that light passes into the reservoir (240) is reflected, passes back through the reservoir and is detected by the sensor (360). This approach allows another sensor to monitor the direct output of the light source to account for variability in the intensity of the light source.

The sensor (360) may detect emitted by a light source provided on an opposite side of the reservoir (240), The light may pass through the reservoir vertically, horizontally, laterally, and/or in some other axis. Vertical orientation has some advantages is settling of the species being detected is a concern in the time frame that the solution remains in the reservoir. In an example, the reservoir includes a pump, impeller, and/or similar component to induce circulation of the fluid in the reservoir.

Other types of sensors (360) may also be used, either independently of a light sensor and/or in combination. An impedance sensor may be used. An advantage of impedance sensors is that the electrodes may be used for multiple purposes. For example, a pair of electrodes may be used to heat the fluid, agitate the fluid using convection, agitate the fluid using dielectric breakdown of a component of the fluid (e.g., hydrolysis), concentrate the fluid by heating and evaporation and/or hydrolysis, etc.

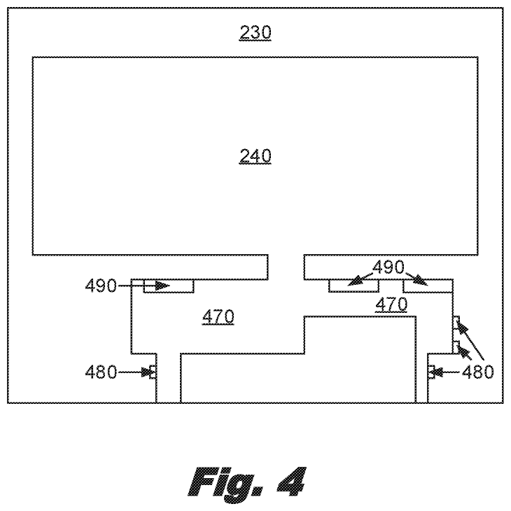

FIG. 4 shows a cross-sectional view through a pair of nozzles of two different firing chambers in an example of an ejection device consistent with the present specification. In FIG. 4 the two nozzles are oriented downward (toward the label FIG. 4). FIG. 4 shows an ejection device (230) with a reservoir (240), The reservoir provides fluid to two firing chambers (470). The firing chamber (470) includes a heater (490) and an electrode (480). The electrode (480) is located in the nozzle. However, other locations of the electrode (480) are possible. Ideally, the volume of fluid in the firing chamber (470) that is ejected as a fired droplet is between the electrode (480) and the heater (470). In this case, an impedance measurement between the electrode (480) and the heater (470) can be used to detect the presence or absence of a cell in the volume of fluid to be ejected as a droplet. A number of different electrode (480) and heater orientations will provide adequate coverage.

The other firing chamber (470) (on the right side of FIG. 4) has two different associated heaters (490). Firing the first heater will produce a droplet of a first size being ejected from the nozzle. Firing the second heater will produce a droplet of a second size being ejected from the nozzle. The two firing chamber (470) are also different sizes, allowing them to generate different sized droplets. The other firing chamber (470) includes multiple electrodes (480). The use of multiple electrodes (480) in the firing chamber (470), nozzle, and the feed to the firing chamber may allow tracking of a cell into the firing chamber; this in turn may allow control the ejection device (230) to fire droplets until the ejection device (230) has fired a droplet containing a cell into the well on the well plate. This may allow more efficient loading of the well plate.

While narrower feed channels to the firing chambers (470) present some design challenges with managing firing bubbles. Such designs can also facilitate detection on monitoring of a cell or cells as they advance toward the firing chamber (470).

It will be appreciated that, within the principles described by this specification, a vast number of variations exist. It should also be appreciated that the examples described are only examples, and are not intended to limit the scope, applicability, or construction of the claims in any way.

What is claimed is:

1. A method of sequencing multiple nucleic acid sequences, comprising:
estimating a concentration of cells in a solution; and
depositing a volume of the solution into a well on a well plate using an ejection device, the amount of solution selected to contain no more than a single cell.

2. The method of claim 1, further comprising depositing lysing reagent into the well using the ejection device.

3. The method of claim 1, wherein the deposited volume is 1% to 20% of an inverse of the estimated concentration for a single cell.

4. The method of claim 3, wherein the deposited volume is 8% to 12% of an inverse of the estimated concentration for a single cell.

5. The method of claim 1, wherein the deposited volume is from 5 to 100 picoliters.

6. The method of claim 1, wherein the deposited volume is from 10 to 30 picoliters.

7. The method of claim 1, further comprising, removing reacted reagent from the well.

8. The method of claim 7, wherein removing the reacted reagent from the well is performed simultaneously on a plurality of wells.

9. The method of claim 1, further comprising diluting the solution to obtain a desired deposition volume prior to depositing the volume of the solution using the ejection device.

10. The method of claim 1, further comprising concentrating the solution to obtain a desired deposition volume prior to depositing the volume of the solution using the ejection device.

11. A system for sequencing a plurality of nucleic acid sequences, the system comprising;
an ejection device comprising a reservoir, the reservoir to hold a solution containing a plurality of cells, the plurality of cells containing different respective nucleic acid sequences to be sequenced; and
a controller to receive a concentration of cells in the solution and to modify the deposited volume of the solution by the ejection device based on the concentration of cells in the solution.

12. The system of claim 11, wherein the ejection device includes a plurality of sizes of nozzles, the plurality of sizes of nozzles to eject different sizes of droplets of the solution.

13. A system for sequencing multiple nucleic acid sources, the system comprising:
an ejection device with a reservoir;
a sensor to detect absorption of light passing through the reservoir; and
a controller to calculate a cell density in a solution in the reservoir from an output of the sensor,
wherein the ejector device receives solution from the reservoir and ejects solution as droplets, each droplet sized to contain at most a single cell, the droplet size being determined by the controller,

14. The system of claim 13, further comprising a plurality of firing chambers of the ejector device, the plurality of firing chambers sized to produce ejected droplets with different volumes.

15. The system of claim 13, further comprising an impedance sensor to detect a cell in a firing chamber of the ejector device.
